(19) United States
(12) Patent Application Publication
(10) Pub. No.: US 2003/0004561 A1
(43) Pub. Date: Jan. 2, 2003

(54) Peeling sheath for self-expanding stent

(76) Inventors: Steve Bigus, San Jose, CA (US); Brent Belding, Los Gatos, CA (US); Suzanne Wallace George, Boston, MA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/894,995

(22) Filed: Jun. 28, 2001

(51) Int. Cl. A61F 2/06

(52) U.S. Cl. 623/1.12

(57) ABSTRACT

A system which is capable of accurately treating an affected area in a body lumen. The system includes a catheter which is positionable in the body lumen at the treatment site and includes an interventional device such as a self-expandable stent which may be deployed in the blood vessel at the treatment site. The system also includes an expansion retention member, adapted to be extended about the interventional device. The system further includes an extendable member, adapted to be extended about the expansion retention member and the interventional device, for delivery of the interventional device to the treatment site, and to be retractable from extending about the expansion retention member for enabling the interventional device to expand at the treatment site.

### BACKGROUND OF THE INVENTION

The present invention relates generally to systems for repairing or treating body lumens, and more particularly to a system which can be used when an interventional procedure is being performed in a stenosed or occluded region of a blood vessel. The system of the present invention is particularly useful when performing stenting procedures in critical vessels, such as the carotid arteries.

A variety of non-surgical interventional procedures have been developed over the years for opening stenosed or occluded blood vessels in a patient caused by the build up of plaque or other substances on the walls of the blood vessel. Such procedures usually involve the percutaneous introduction of the interventional device into the lumen of the artery. One widely known and medically accepted procedure is balloon angioplasty in which an inflatable balloon is introduced within the stenosed region of the blood vessel to dilate the occluded vessel. The balloon catheter is initially inserted into the patient's arterial system and is advanced and manipulated into the area of stenosis in the artery. The balloon is inflated to compress the plaque and press the vessel wall radially outward to increase the diameter of the blood vessel.

Another procedure is laser angioplasty which utilizes a laser to ablate the stenosis by super heating and vaporizing the deposited plaque. Atherectomy is yet another method of treating a stenosed blood vessel in which a cutting blade is rotated to shave the deposited plaque from the arterial wall. A vacuum catheter is usually used to capture the shaved plaque or thrombus from the blood stream during this procedure.

In another widely practiced procedure, the stenosis can be treated by placing an expandable interventional device such as an expandable stent into the stenosed region to hold open and sometimes expand the segment of blood vessel or other arterial lumen. Stents are particularly useful in the treatment or repair of blood vessels after a stenosis has been compressed by percutaneous transluminal coronary angioplasty (PTCA), percutaneous transluminal angioplasty (PTA) or removal by atherectomy or other means. Stents are usually delivered in a compressed condition to the target site, and then are deployed at the target location into an expanded condition to support the vessel and help maintain it in an open position.

In the past, stents typically have fallen into two general categories of construction. The first type of stent is expandable upon application of a controlled force, often through the inflation of an expandable member such as an expandable balloon in a dilatation catheter which, upon inflation of the balloon or other expansion means, expands the compressed stent to a larger diameter to be left in place within the artery at the target site. The second type of stent is a self-expanding stent formed from, for example, shape memory metals or super-elastic nickel-titanum (NiTi) alloys, which will automatically expand from a compressed state when the stent is advanced out of the distal end of the delivery catheter into the body lumen. Such stents manufactured from expandable heat sensitive materials allow for phase transformations of the material to occur, resulting in the expansion and contraction of the stent.

Self-expanding stents are typically delivered to an interventional procedure site for deployment thereof mounted on a delivery system and constrained in the sheath, to prevent the elastic nature of the self-expanding stent from causing it to expand prematurely. Once in position at the interventional procedure site, the sheath is retracted, enabling the stent to expand and deploy. However, there are sometimes problems associated with the retraction of the sheath for enabling accurate deployment of the self-expanding stent. When the sheath is retracted during stent deployment, axial forces are generated in the catheter when one end of the stent is fully open and the other end is still constrained. The stent is biased to slip out from under the sheath and finish deploying. An abrupt shortening that occurs as the stent deploys also generates axial forces. These axial forces can cause the stent to move in the distal direction during deployment and not properly cover the repair site.

Additionally, where a conventional delivery system including an inner member about which a stent is mounted and an outer sheath covering the stent, is employed to deliver the stent at a repair site, forces are created in components of the conventional delivery system which make it more difficult to accomplish the repair procedure and which add to the complexity of the delivery system. That is, in conventional systems, the outer sheath is withdrawn relative to the inner member, which thereby subjects the outer sheath to tension forces and the inner member to compression forces. In order to withstand such forces, the components of conventional systems must embody a relatively high degree of structural integrity and strength. Unfortunately, providing components of conventional delivery systems with required structural integrity and strength generally results in decreasing the flexibility of the components. Moreover, as retraction forces increase, the inner member stiffness must increase to prevent buckling and the outer sheath must be stiffer to prevent elongation. Bond strengths between components may also need to be increased and the sheath may need to be thicker. Such requirements result in the delivery system being less deliverable.

Traditional self-expanding stent single-layer sheathing systems must slide over the stent to allow for deployment. Frictional laws state that the amount of force required to pull the sheath over the stent is equal to the expansion force of the stent multiplied by the coefficient of friction between the stent and the sheath. Thus, increasing the stent strength (e.g., expansion force) results in higher sheath pull forces. Similarly, as the stent becomes rougher (e.g., higher coefficient of friction), the sheath pull forces increase. Additionally, due to the stated interaction between a stent and a conventional delivery sheath, there is a limit to the extent to which the stent can be compressed for delivery through body lumens. That is, the greater the compression of a self-expanding stent, the greater the outwardly directed radial forces which are generated by the stent. However, such radial forces make it more difficult to withdraw a sheath of a conventional delivery system and thus, have a bearing on the degree to which the profile of a delivery system can be minimized.

Accordingly, what has been needed is a reliable system and method for delivering an interventional device for treating body lumens which improve the accuracy of stent deployment. The system and method should be capable of enabling the interventional device to expand, while precisely placing the device and creating a smooth transition between constrained and unconstrained portions of the device. Moreover, such a system should be relatively easy to deploy and remove from the patient's vasculature. Further, the system should embody high flexibility and structure aimed at facilitating a higher degree of compression of a stent for delivery at a repair site. The invention disclosed herein satisfies these and other needs.

### SUMMARY OF INVENTION

The present invention provides a system and method for treating body lumens. The present invention is particularly useful when performing an interventional procedure in vital arteries, including the main blood vessels leading to the brain or other vital organs. As a result, the present invention provides the physician with a higher degree of confidence that an entire repair site will be treated, and that healthy tissue will not be adversely affected by the treatment procedure. The present invention enables an interventional procedure to be performed in a body lumen in a manner such that axial movement of an interventional device is prevented during retraction of a sheath extending thereabout.

Moreover, the present invention provides structure facilitating increased radial compression of an interventional device and the minimization of the profile of a delivery system embodying the sheath of the present invention. Further, by reducing forces between an interventional device and the sheath during deployment, components of the system embody higher flexibility and require less structural strength and integrity. Additionally, withdrawing the sheath of the present invention causes reduced trauma to an interventional device.

In one aspect of the present invention, the system includes a catheter for positioning in a blood vessel at an interventional procedure site and an interventional device located at a distal end portion of the catheter. The system further includes an extendable member adapted to receive the interventional device and to be retractable relative thereto, and an expansion retention element for preventing axial movement of the interventional device during retraction of the extendable member.

In one embodiment of the present invention, the system includes a catheter, including an elongated shaft having a distal end portion adapted to be positioned in a blood vessel at an interventional procedure site. An interventional device configured to move between collapsed and expanded positions is supported on the distal portion of the elongated shaft. An extendable member is further included and is adapted to be extendable about the interventional device and to be retractable relative thereto. An expansion retention member, adapted for preventing axial movement of the interventional device during retraction of the extendable member facilitates precise deployment of the interventional device.

In another embodiment of the present invention, it is contemplated that the system include a lubricant between an extendable member sheath and an expansion retention member sheath. The extendable member and the expansion retention member are adapted to be extendable about the interventional device for delivery at the interventional procedure site, and to be retractable for enabling the interventional device to gradually expand at the interventional procedure site. The application of the lubricant between the extendable member and the expansion retention member is intended to further prevent axial movement of the interventional device during retraction of the extendable member and the peeling away of the expansion retention member by reducing the amount of friction between the dual-layer sheath, and consequently reducing the amount of pulling force required.

Other features and advantages of the present invention will become more apparent from the following detailed description of the preferred embodiments of the invention, when taken in conjunction with the accompanying exemplary drawings.

### BRIEF DESCRIPTION OF THE DRAWINGS

FIG. 1 is a partial cross-sectional view, depicting the system of the present invention disposed at a treatment site within a body lumen of a patient;

FIG. 2 is a partial cross-sectional view, depicting the system of FIG. 1, wherein an extendable member is partially retracted and an interventional device is in a partially expanded condition;

FIG. 3 is a partial cross-sectional view, depicting the system of FIG. 2 with the interventional device in a fully expanded condition.; and

FIG. 4 is a partial cross-sectional view, depicting the system of FIG. 3 being retracted from the treatment site.

### DETAILED DESCRIPTION OF THE PREFERRED EMBODIMENTS

The present invention is directed to an improved system and method for efficiently and effectively enabling a therapeutic interventional procedure to be performed in a body lumen. The system includes structure which is intended to prevent axial movement of an interventional device during retraction of an extendable member and to accomplish the peeling away of an expansion retention member for enabling accurate deployment of the interventional device at a treatment site. The system also includes structure facilitating minimizing profile as well as trauma to an interventional device being delivered thereby. Moreover, by reducing forces between an interventional device and the system, the structure of the system embodying the present invention can have higher flexibility and require less structural integrity and strength. In one aspect, the system is configured to facilitate the delivery of a self-expandable interventional device at an interventional procedure site. The embodiments of the improved system and method are illustrated and described herein by way of example only and not by way of limitation. While the present invention is described in detail as applied to the carotid arteries of the patient, those skilled in the art will appreciate that it can also be used in other body lumens as well, such as the coronary arteries, non-coronary arteries, renal arteries, saphenous veins and other peripheral arteries or organs.

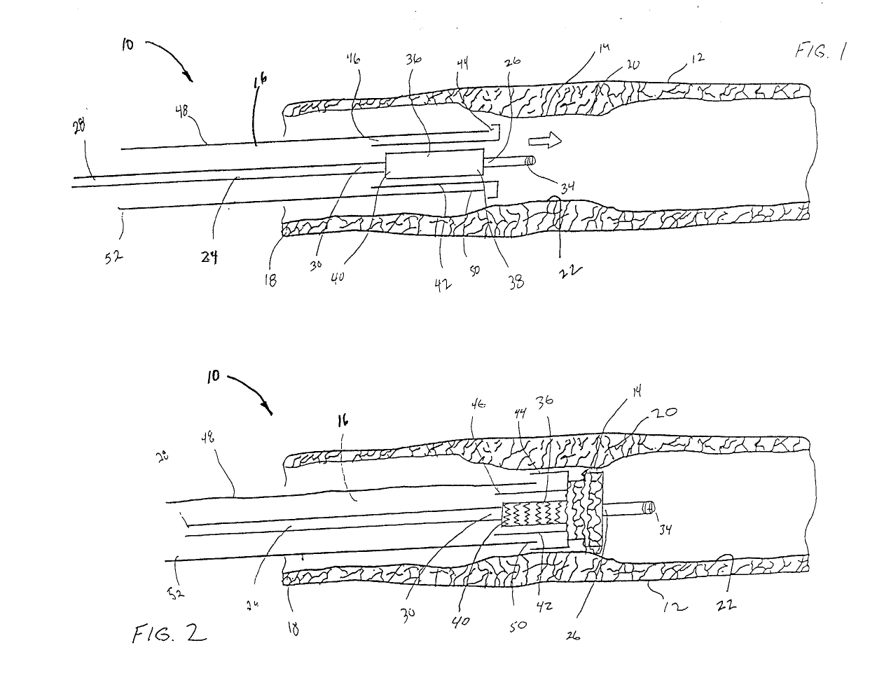

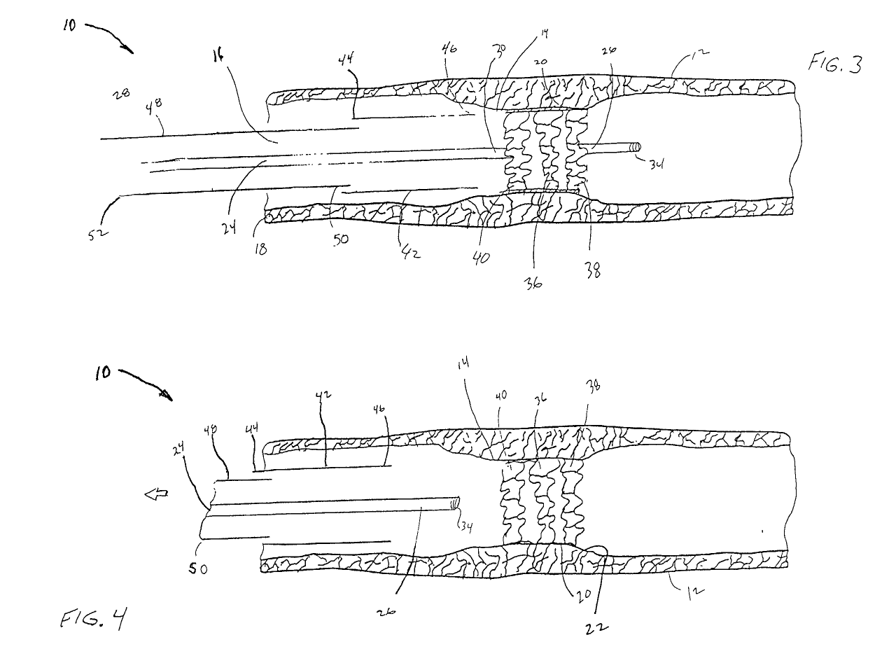

Referring now to the drawings, wherein like reference numerals denote like or corresponding parts throughout the drawing figures, and in particular to FIGS. 1-4, an exemplary system 10 is depicted for facilitating the performance of an interventional procedure in a blood vessel 12 at an area of treatment 14. As shown in FIGS. 2-4, the system 10 includes a catheter 16, and is contemplated to be placed within a carotid artery 18 or other blood vessel of the patient. In one aspect, the system 10 may be guided into position by a guide wire 34. The area of treatment 14 may suffer from atherosclerotic plaque 20 which built up against the inside wall 22 of the carotid artery 18. As a result, blood flow is diminished through this area. The catheter 16 further includes an elongated shaft 24 having a distal end 26, a proximal end 28 and an internal lumen adapted to receive the guide wire 34.

The therapeutic interventional procedure includes implanting an interventional device 36 at the treatment site 14, to press the build-up of plaque 20 against the inside wall 22 and thereby restore sufficient flow of blood to the downstream vessels leading to the brain. The interventional device 36 not only helps increase the diameter of the occluded area, but may help prevent restenosis in the area of treatment 14. In one embodiment, the interventional device 36 is adapted to be positioned upon the distal portion of the catheter shaft 26, and to be expanded and deployed at the treatment site 14. The device 36 is expandable in a direction generally transverse to an axial dimension thereof. The interventional device 36 may embody for example, a self-expandable stent, the elastic nature of which provides self-expansion absent constraint. The self-expandable stent 36 includes a distal end 38 and a proximal end 40.

A generally tubular expansion retention member 44 constrains the self-expandable stent 36 and is configured about the self-expandable stent 36 to prevent expansion prior to delivery to the treatment site. In one aspect, the expansion retention member 42 includes a thin material having an inner surface and outer surface. Further, to minimize the profile of the delivery system, when assembled, the proximal portion of the retention member 44 extends only to the proximal end 40 of the stent 36.

The system 10 further includes a generally tubular member 48 which extends about the expansion retention member 42 and the self-expandable stent 36. The extendable member 48 is moveable longitudinally relative to the self-expandable stent 36. The extendable member 48 defines an outer sheath having an inner surface and an outer surface. The extendable member 48 and the expansion retention member 42, in combination, form a dual-layer sheath assembly which prevents axial movement of the self-expandable stent 36 with respect to the distal region 26 of the catheter elongated shaft 24 during retraction of the outer sheath 48.

In the embodiment of the invention illustrated in FIGS. 1-4, the expansion retention member 44 and the extendable member 48 are bonded together. Alternatively, the two members 44,48 can embody one material or structure to thereby visicate the need for a bond. The inner sheath 42 extends past the distal end 50 of the outer sheath 48 and is folded back proximally and over the outer sheath 48 to allow for bonding between the outer surface of the outer sheath and a folded portion of the inner sheath 42 distal end 44. The proximal end 46 of the inner sheath 42 could be bonded to the inner member or elongated shaft 24 or could be free floating (i.e., not permanently bonded to any structure).

In a preferred embodiment, the outer sheath 48 extends to the proximal-most portion of the system 10, so it can be accessed by a physician outside of a patient's anatomy. It is contemplated that a handle or other control-enabling device is attached to the proximal end of the outer sheath to accomplish longitudinal movement of the system. As stated, the inner sheath 42 extends only to the proximal end 40 of the self-expanding stent 36. The self-expandable stent 36 is deployed at the interventional site by retracting the outer sheath 48 proximally which in turn retracts the inner sheath 42. The proximal retraction of the inner sheath 42 distal end 44 results in a peeling motion away from the self expandable stent 36, allowing the stent 36 to gradually expand to its unconstrained diameter. The peeling movement of the inner sheath 42 from the stent 36 reduces the jumping effect traditionally caused by sliding a single-layer sheathing system over the stent for deployment. Frictional laws state that the amount of force required to pull the sheath over the stent is equal to the expansion force of the stent multiplied by the coefficient of friction between the stent and sheath:

Sheath Pull Force=(Coefficient of Friction)*(Stent Expansion Force)

The force required to retract the dual-layer sheath must be kept to a minimum such that the retraction force does not exceed the bond strength between the inner sheath 42 and the outer sheath 48 or other junctions within the catheter. Additionally, the force required to pull back the outer sheath 48 must be clinically acceptable, such that the physician does not have to apply an uncomfortable amount of force to retract the outer sheath 48.

The present invention reduces the frictional forces that are present in the catheter system by changing the mechanics of the stent/sheath interaction. Instead of sliding over the stent 36, the inner sheath 42 in this invention peels away from the stent 36. Because the inner sheath 42 is not sliding over the stent 36, the coefficient of friction between the sheath 42 and stent 36 is no longer critical, and the force required to pull back the dual-layer sheath is reduced. Thus, the components of the system can have greater flexibility because less strength is required to accomplish the relative movement between the sheaths 42,48 and inner member or shaft 24. Additionally, the stent 36 can be crimped within the sheaths 42, 48 to a greater degree because less force is required to be applied to the sheaths 42, 48 to release the stent 36. That is, increased outwardly directed radial force generated by the more highly compressed stent 36 can be better accommodated by the peeling sheath design. In another preferred embodiment of the invention, a further reduction of friction may be achieved by applying a lubricant between the inner sheath and outer sheath layers. This reduction in friction may be very important if the stent is coated with a drug/polymer/or active substance. The friction generated from a translational sheath retraction may remove the stent coating whereas a sheath that peels away may not damage the fragile stent coating. Therefore, the stent is exposed to less trauma using the system of the present invention. In use, as illustrated in FIGS. 1-4, the system 10 may be placed within a patient's vasculature utilizing any one of a number of conventional methods. In one preferred method of positioning, the catheter elongated shaft support region 30, the stent 36 supported thereon, the inner 42 and the outer sheaths 48 extending thereabout, may be placed in a blood vessel 12 utilizing the catheter 16 and manipulated by the physician to the area of treatment 14. The outer sheath 48 is then retracted from extending about the stent 36, so as to peel away the inner sheath 42 from the stent 36 and enable the stent 36 to expand at the treatment site 14. As the outer sheath 48 is retracted, the peeling back of the inner sheath results in a gradual stent expansion in a manner preventing axial movement of the stent 40 during retraction of the outer sheath 48.

Referring now to FIG. 2, as the outer sheath 48 is pulled back, the inner sheath 42 lags slightly behind the outer sheath. This results in a portion of the thin flexible layer 42 being folded over itself at the distal end 46, whereby a gradual transition is provided between the constrained and unconstrained diameters of the stent 36. The unconstrained portion of the self-expanding stent 36 expands in the inside wall 22 of the treatment site 14 and stabilizes the stent 36 during continued deployment. This tapering transition results in a more gentle deployment of the stent 36, and a reduction in the stent 36 jumping.

Materials adapted for use in the inner sheath 42 include a thin flexible material, that may include Plexar, Primacor, or ePTFE. Further, the inner sheath 42 and outer sheath 48 bond and various other components may be joined by suitable adhesives such as acrylonitrile based adhesives or cyanoacrylate based adhesives. Heat shrinking, heat bonding or laser bonding may also be employed where appropriate. Plastic-to-plastic or plastic-to-metal joints can be effected by a suitable acrylonitrile or cyanoacrylate adhesive. Variations can be made in the composition of the materials to vary properties as needed.

It should be appreciated that the dual-layer sheath assembly of the present invention is capable of being positioned about a self-expandable stent 36. However, other forms of treatment or repair devices may be utilized with the present invention without departing from the spirit and scope of the invention. For example, the treatment device may further embody other forms of material and mesh configurations and may require a radial force to accomplish expansion. Additionally, while the sheaths are shown being generally tubular, the dual-layer sheath assembly can be formed in any one of a number of different shapes depending upon the need.

In view of the foregoing, it is apparent that the system and method of the present invention enhances substantially the effectiveness of performing treatments by preventing axial movement of a treatment device during deployment. Further modifications and improvements may additionally be made to the system and method disclosed herein without the departing from the scope of the invention. Accordingly, it is not intended that the invention be limited, except as by the appended claims.

What is claimed:

1. A system for enabling an interventional procedure to be performed in a blood vessel at a treatment site, comprising:
an elongated shaft which includes a distal end portion adapted to be positioned in a blood vessel at a treatment site;
an expandable interventional device positioned on the distal end portion of the elongated shaft;
an expansion retention member adapted to receive the expandable intervention device in a compressed configuration, the retention member having a first end portion, a second end portion and a cross-sectional wall thickness; and
an extendable member attached to the first end portion and configured about the second end portion when the system is assembled for use, the extendable member providing a gradual tapering transition when the interventional device is being deployed at the procedure site.

2. The system of claim 1, wherein the expansion retention member is an inner sheath comprised of a thin flexible material.

3. The system of claim 1, wherein the extendable member is an outer sheath.

4. The system of claim 1, the expandable interventional device comprising a stent.

5. The system of claim 1, wherein the expansion retention member is folded over a distal end of the extendable member and retraction of the extendable member peels the expansion retention member from the expandable interventional device.

6. The system of claim 5, further comprising an element that facilitates retraction of the outer sheath.

7. The system of claim 5, the elongated shaft includes a proximal end, and the outer sheath includes a distal end, further comprising an element for enabling control of distal movement of the outer sheath from the proximal end of the catheter elongated shaft, wherein the control-enabling element is adapted to be connected to the proximal end of the outer sheath.

8. The system of claim 1, further comprising a lubricant configured between the expansion retention member and the extendable member.

9. The system of claim 1, wherein the expansion retention member is an inner sheath comprised of a thin flexible material and having a tubular configuration.

10. The system of claim 1, wherein the extendable member is an outer sheath having a generally tubular configuration.

11. The system of claim 1, the interventional device further comprising a self-expanding stent, the elastic nature of which enables self-expansion thereof absent constraint.

12. The system of claim 1, wherein the expansion retention member is bonded to the extendable member.

13. The system of claim 1, wherein a partially deployed interventional device is constrained only by a single layer of the expandable member and a double layer of the expansion retention member.

14. The system of claim 1, wherein the extendable member is moveable longitudinally relative to the shaft.

15. The system of claim 13, wherein the interventional device requires an external force to cause it to expand radially.

16. A method for treating a body lumen using a system including a catheter having an expansion retention member having a first end connected to an external surface of an extendable member and a second end removably contained within an exterior of the extendable member, the expansion retention member operating to releasably contain a treatment device in a constrained configuration and having a portion forming two layers, comprising;
placing the system within a body lumen;
advancing the system to a repair site; and
partially releasing the treatment device by configuring a first portion of the treatment device within both the expansion retention member and the extendable member, a second portion of the treatment device within only two layers of the expansion retention member and a third portion of the treatment device in contact with the body lumen such that a gradual tapered transition is provided by the expansion retention member and extendable member.

17. The method of claim 16, wherein the treatment device is a self-expanding stent.

18. The method of claim 16, further comprising completely disengaging the treatment device from the catheter.

19. The method of claim 18, wherein the catheter further includes a lubricant facilitating the disengagement of the treatment device from the catheter.

20. The method of claim 16, further comprising releasing the treatment device in a manner avoiding jumping of treatment device from the catheter.
